(19) United States
(12) Patent Application Publication
(10) Pub. No.: US 2026/0311814 A1
(43) Pub. Date: Oct. 8, 2026

(54) OPTIMIZING CONFIGURATION OF INPUT PARAMETERS TO A BIOMEDICAL FOUNDATIONAL MODEL

(71) Applicant: International Business Machines Corporation, Armonk, NY (US)

(72) Inventors: Uri Kartoun, Cambridge, MA (US); Akira Koseki, Yokohama-shi (JP); Natalia Mulligan, Dublin (IE); Joao H. Bettencourt-Silva, Dublin (IE); VIBHA Anand, Chestnut Hill, MA (US)

(21) Appl. No.: 19/171,876

(22) Filed: Apr. 7, 2025

(51) Int. Cl. G16B 40/00; G16B 5/00

(52) U.S. Cl. CPC G16B 40/00; G16B 5/00

(57) ABSTRACT

A method optimizes the configuration parameters of a biomedical foundation model for single-cell genetic analysis. Different sets of configuration parameter values are evaluated by ranking genes based on their association strengths with a given cell type. The deviation of median ranking values across iterative runs, along with the processing times of those runs, is used to select the final configuration parameter set for the biomedical foundation model.

### BACKGROUND

The present application relates generally to computers and computer applications, and more particularly to input parameter configuration optimization for biomedical foundational models.

### BRIEF SUMMARY

The summary of the disclosure is given to aid understanding of a computer system and method of optimizing configuration of input parameters to biomedical foundational model, and not with an intent to limit the disclosure or the invention. It should be understood that various aspects and features of the disclosure may advantageously be used separately in some instances, or in combination with other aspects and features of the disclosure in other instances. Accordingly, variations and modifications may be made to the computer system and/or their method of operation to achieve different effects.

In some embodiments, computer-implemented method includes evaluating sets of values for a set of configuration parameters of a biomedical foundation model, where the evaluating comprises performing an interpretability assessment on a cell type. In some embodiments, the interpretability assessment includes using a set of values from the sets of values as the set of configuration parameters for the biomedical foundation model, running the biomedical foundation model to obtain association strength scores between the cell type and genes. The interpretability assessment also includes ranking the genes by the association strength scores. The interpretability assessment also includes selecting from the ranking, a pre-defined number of top ranked genes and a pre-defined number of additional genes. The interpretability assessment also includes storing the pre-defined number of top ranked genes and the pre-defined number of additional genes. The interpretability assessment also includes repeating by a pre-defined number of times, the using and the ranking in consideration with the pre-defined number of top ranked genes and the pre-defined number of additional genes. The interpretability assessment also includes, for each gene in the pre-defined number of top ranked genes and the pre-defined number of additional genes, determining a median value of rankings determined from the repeating of the using and the ranking the pre-defined number of times. The computer-implemented method also includes iteratively performing the interpretability assessment for each of the sets of values for the set of configuration parameters. The computer-implemented method also includes, for each gene in the pre-defined number of top ranked genes and the pre-defined number of additional genes, determining a standard deviation among median values of rankings that are computed during the interpretability assessment performed iteratively. The computer-implemented method also includes comparing the standard deviation with a standard deviation threshold. The computer-implemented method also includes determining a median running time of the biomedical foundation model for each iteration of the interpretability assessment. The computer-implemented method also includes, based on the comparison of the standard deviation with the standard deviation threshold and the median running time, selecting a final set from the sets of values to use as the set of configuration parameters for running the biomedical foundation model.

In some embodiments, a system including a processor set, a set of one or more computer-readable storage media and program instructions, collectively stored in the set of one or more computer-readable storage media, for causing the processor set to perform the computer operations that include one or more methods described herein are also provided.

In some embodiments, a computer program product comprising a set of one or more computer-readable storage media, and program instructions, collectively stored in the set of one or more storage media, for causing a processor set to perform computer operations that perform one or more methods described herein also may be provided.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1 shows an example of a computing environment, which can implement input parameter configuration optimization in some embodiments.

FIG. 2 is a flow diagram illustrating a method that optimizes a configuration of input parameters to biomedical foundational model in some embodiments.

FIG. 3 is a diagram illustrating a use case example in some embodiments, in which a 16 core processor is used in running the foundation model, where the top 3 ranked genes are shown.

FIG. 4 shows a continuation of the use case shown in FIG. 3, where the top 28^{th}, 29^{th} and 30^{th} ranked genes are shown in some embodiments.

FIG. 5 shows processing time results from use case examples where different byte sizes are used as maximum input length in foundation model iterations in some embodiments.

FIG. 6 shows another use case example, in which an 8 core processor is used in running the foundation model in some embodiments.

FIG. 7 shows continuation of the use case example of FIG. 6, where the results are shown for the 28^{th}, 29^{th} and 30^{th} ranked genes in some embodiments.

FIG. 8 shows processing time results from use case examples where different byte sizes are used as maximum input length in runs a foundation model in some embodiments.

FIG. 9 shows a median processing time comparison between running the foundation model on different types of with different maximum input length configurations in some embodiments.

FIGS. 10A-10B show flow diagrams illustrating a method of optimizing configuration of input parameters to a biomedical foundation model in some embodiments.

### DETAILED DESCRIPTION

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment ("CPP embodiment" or “CPP”) is a term used in the present disclosure to describe any set of one, or more, storage media (also called "mediums") collectively included in a set of one, or more, storage devices that collectively include machine readable code corresponding to instructions and/or data for performing computer operations specified in a given CPP claim. A "storage device" is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits / lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as input parameter configuration optimization code 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

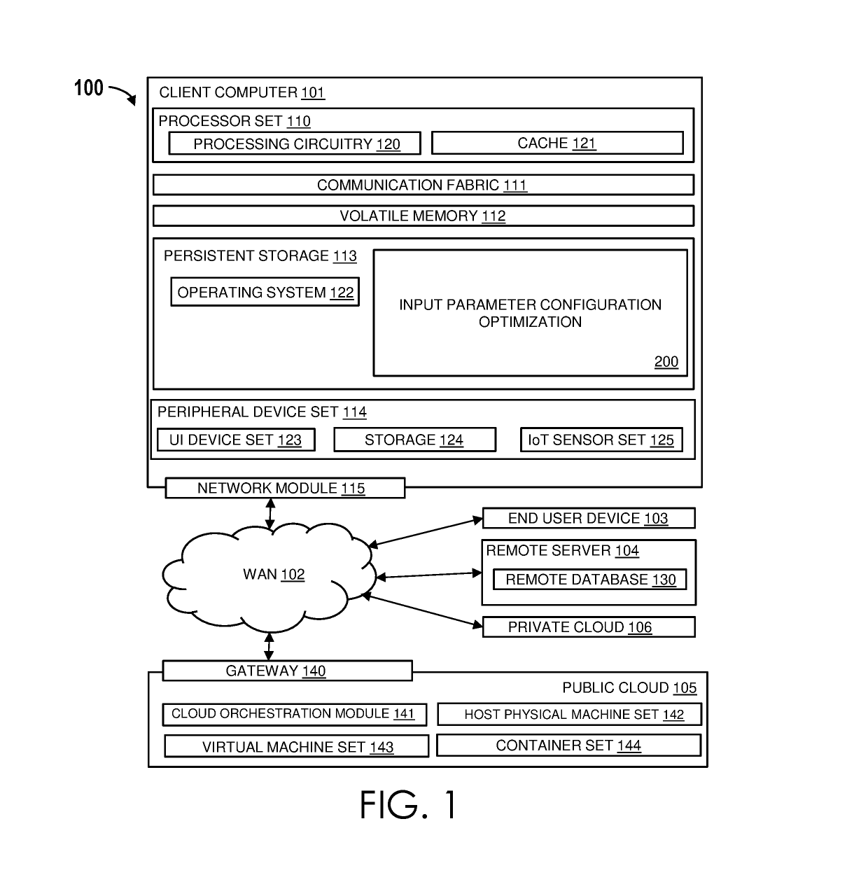

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input / output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

Systems, methods and/or techniques are provided that optimize values of configuration parameters of a biomedical foundation model, e.g., in a single cell genetic analysis. Single-cell analysis facilitates in disease diagnosis, discovery and development of new candidate medications, sequencing technology, cell isolation. Biomedical foundation models are trained, fine-tuned and used in performing gene or single-cell analysis. Using the systems, methods and/or techniques, developers and/or scientists may identify configuration parameters that can save processing time of a computer processor in performing gene and/or single-cell analysis, and also reduce connectivity issues.

Foundation models are large-scale, general-purpose artificial intelligence (AI) models trained on vast datasets, serving as a base for diverse applications, and can be fine-tuned for a wide range of specific tasks. Foundation models have a preprocessing function that processes data used by the foundation models, for example, in training and/or inference phases. An example of a preprocessing function is a data collator.

A biomedical foundation model in inference phase can rank an association level between a gene and a given cell or cell type, e.g., how strongly a gene and a given cell type are associated with one another. This ranking facilitates in discovering what specific genes are associated with a given cell. Such information can then be used in medical applications such as new candidate medication development and/or others. In some embodiments, the system and/or method allows for running such a foundation model in a resource-saving manner. For example, a foundation model run according to a technique disclosed herein takes less time to run, and use less computational resources such as processing power and memory.

A foundation model such as the single-cell Bidirectional Encoder Representations from Transformers (scBERT) is a pretrained deep neural network-based model, based on transformer architecture. In the context of neural network models (e.g., scBERT), a data collator is a component used for processing and organizing data. Using a data collator, a transformer model can show or expose the input data that is significantly larger than the model’s input size. Specifically, in a transformer-based foundation model with limited input length, data collator is responsible for preparing the data in a format suitable for the neural network (NN) consumption techniques (including training, inference and interpretation). Data preparation includes applying processes such as splitting, amending (masking), aggregating, and padding.

Data collators require specifying values of a variety of parameters in a collator object. For example, a collator object may include attributes such as “pad to multiple”, “max length”, “sequence order” and “batch size”. Values are assigned to those attributes, e.g., “pad to multiple=16”, “max length=512”, “sequence order=random”, and “batch size=64”. Based on the assigned values of the attributes, the collator preprocesses data to be input to a foundation model.

The “pad to multiple” attribute specifies that the input sequences should be padded to a length that is a multiple of the specified parameter. Padding is a technique used to ensure that all sequences in a dataset have the same length.

The “max length” attribute sets the maximum allowed length for input sequences. Sequences longer than this value are truncated to fit within the specified length. Setting a maximum length helps control memory usage.

The “sequence order” attribute (e.g., whose values can include sequential, sorted, reverse, random) indicates the order of the sequences in the dataset. Randomizing, for example, can help prevent the AI model from learning spurious patterns based on the order of the data.

The “batch size” attribute is responsible for how many fragments of the same cell are shown to the model.

In a typical single-cell genetic analysis, a computer-implemented process may access a large database that contains cell-related data (e.g., Panglao) and also access a single cell pre-trained foundation model such as scBERT. Optionally, the computer-implemented process may incorporate a domain-specific data (e.g., scp253). Then, the computer-implemented process applies fine tuning to the foundation model. The computer-implemented process also specifies parameters (e.g., as collator object discussed above) for configuring the data to be input to the fined-tuned model. For instance, a value for “pad to multiple” can be specified as one of “8”, “16”, “32”, or another value; a value for “max length” can be specified as one of “128”, 256”, “512”, or another value that specifies a byte size for the maximum length of input data; a value for “sequence order” can be specified as one of “none”, “random”, or another value that specifies a type of sequence order. The data collator pre-processes the data to be input to the foundation model, according to the values of the collator object attributes. The pre-processed data is then input to the foundation model (e.g., that is fine-tuned), where the foundation model outputs its results or finding. For example, cells can be classified and the classification decision can be explained using one or more of the interpretation techniques such as CAPTUM or LIME. Also, for any given cell, the foundation model may calculate for each gene association strength. Using the existing literature and/or domain expertise, the computer-implemented process may also include assessing discoverability.

A task of a data collator for neural network is to transform input data into a shape consumable by the neural network such that the model performance of the neural network is optimized. This task or shaping of input data can be achieved by different means, e.g., by implementing effective masking. A challenge of a data collator for gene data in a cell context is to form input data of a smaller than actual input length without losing model performance and/or accuracy.

Selecting values of data collator attributes arbitrarily to reduce input data dimension may affect the findings or results of the foundation model, which for example, may lead to a compromise in interpretability in gene-cell classification and/or performance speed in which a task may take multiple days to run rather than hours, and/or result in incomplete or break in the task.

In some embodiments, a system and/or method allows for specifying the attribute values of data collator attributes that can contribute to faster processing of the foundation model, substantially preserve the interpretability level, and also assess interpretability discrepancy.

In some embodiments, the system and/or method is applicative specifically to a single cell genetic analysis, in which grid search approach is not applicative. In some embodiments, the system and/or method combines top and bottom attribution scores of genes to assess if interpretability is preserved while consuming less computational resources.

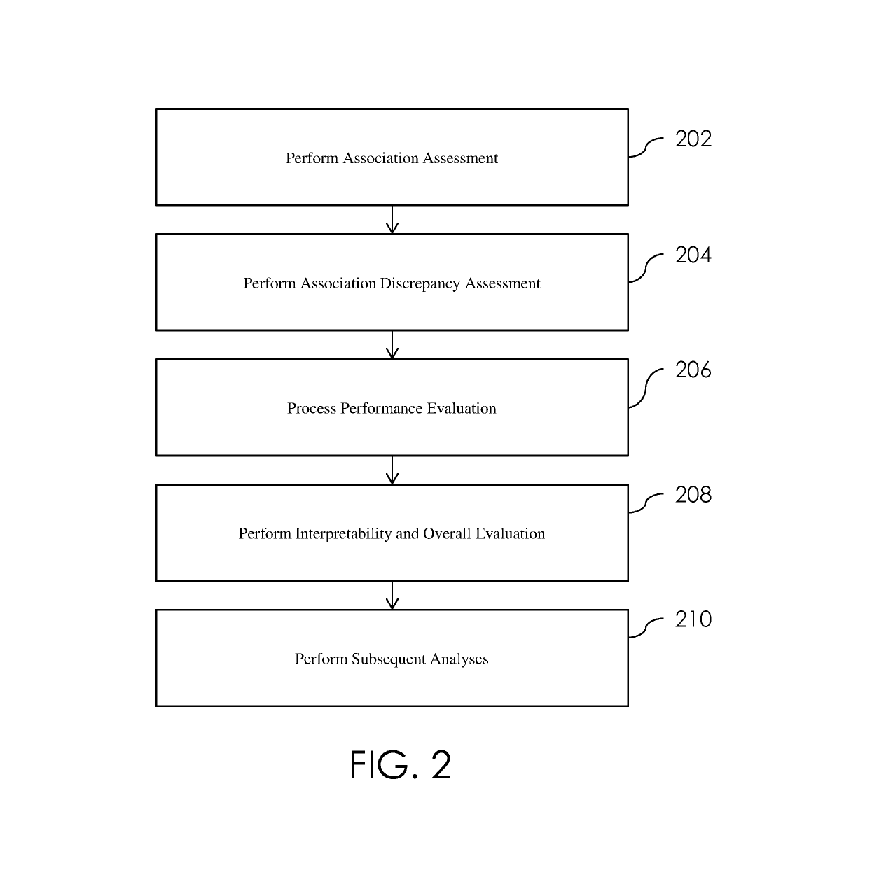

FIG. 2 is a flow diagram illustrating a method that optimizes a configuration of input parameters to biomedical foundational model in some embodiments. The steps are performed for each set of configuration parameters, e.g., the collator object described above. At 202, association assessment is performed. Association assessment includes the following processing. For a given one cell type and genes (e.g., approximately 20,000), a computer-implemented process applies an interpretability iteration to assess associations between genes and a cell type. Applying an interpretability iteration includes running a foundation model by feeding the foundation model the given one cell type and genes. The foundation model determines associations between genes and cell types. Based on the interpretability iteration, the computer-implemented process determines top n1 ranked genes, where n1 is an integer, e.g., top 1, 2, and 3 genes having closest associations to the cell type. The computer-implemented process also determines n2 genes with lower ranks, where n2 is an integer, e.g., top 28^{th}, 29^{th} and 30^{th}ranked genes. The computer-implemented process stores the six ranked genes, e.g., top 1, 2, 3 and top 28, 29, 30. By way of example, n1 is 3, and those top three genes are ZG16, FCGBP, and MUC2; n2 is 3, and those 28^{th}, 29^{th} and 30^{th} ranked genes are MT-CO3, MTRNR2L1, and MUC1. The computer-implemented process applies additional interpretability iterations, for example, 9 more.

In each iteration is stored a rank for each gene. Considering the ranks (e.g., of the 10 iterations), the computer-implemented process calculates median and quartile values (e.g., Q1, Q3) for each of the ranks associated with the genes (6 genes in this example).

At 204, association discrepancy assessment is performed. Association discrepancy assessment includes performing the following for a set of configuration parameters. For each of the genes considered, the computer-implemented process calculates a standard deviation, and assesses a discrepancy of the standard deviation from a pre-defined threshold value.

At 206, performance evaluation is processed. Processing performance evaluation includes performing the following for the set of configuration parameters. For each iteration, the computer-implemented process measures processing time, and calculate a median processing time and quartile values (e.g., Q1, Q3).

At 208, interpretability and overall evaluation is performed. Interpretability and overall evaluation includes determining or evaluating whether a set of configuration parameters resulted in a better processing performance, and did not affect interpretation conclusion.

At 210, subsequent analyses may be performed. A subsequent analysis may include using the optimal configuration parameters, e.g., determined at 208. Such parameters can be configured automatically or manually.

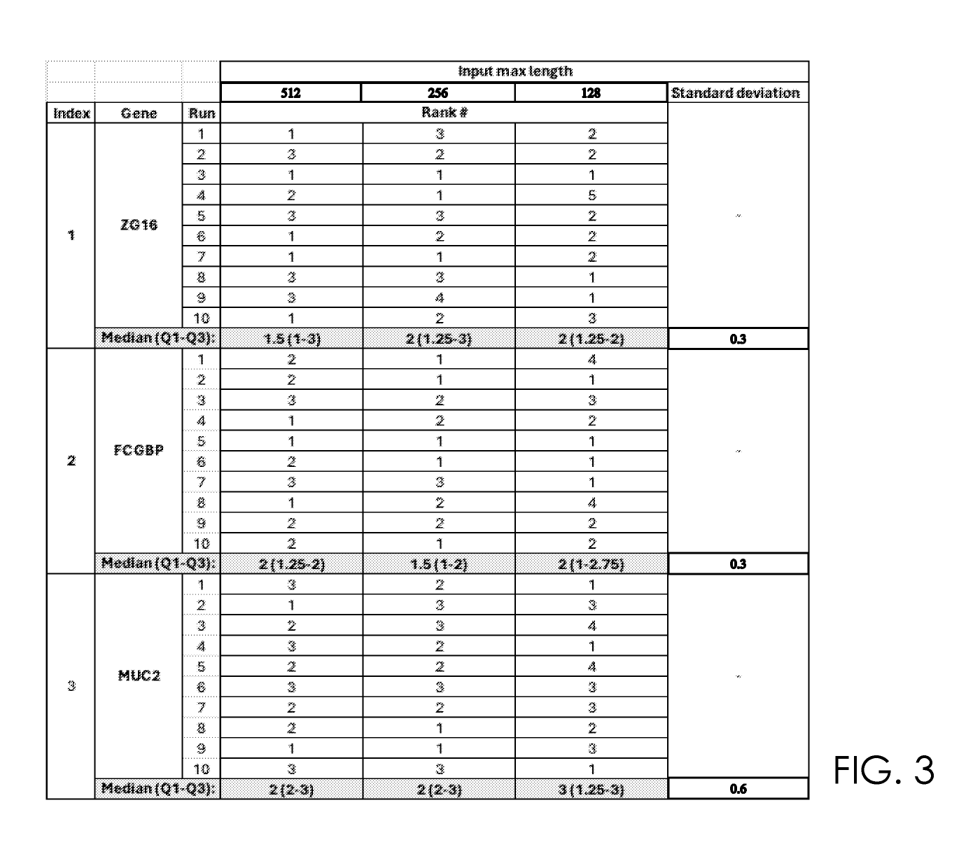

FIG. 3 is a diagram illustrating a use case example in some embodiments. As described above, a data collator configures and presents an input data to a foundation model in an optimal manner so that the foundation model runs efficiently. The use case example shows results obtained from running a foundation model that determines which genes are most associated with a given cell. In this use case example, the given cell is a goblet cell. There can be a large number of genes that can be compared for associations. For example, there can be 20,000 or a smaller number of genes. The use case example shows top 3 different genes and their association rankings obtained using a foundation model. In the first run, the foundation model identified or determined that “ZG16” gene was ranked 1^{st} in association strength, “FCGBP” gene was ranked 2^{nd} in association strength, and “MUC2” gene was ranked 3^{rd} in association strength, when the input length was 512 bytes. Nine additional iterations are performed, where the foundation model ran nine additional times. The results show that the rankings of those 3 genes toggle between 1^{st}, 2^{nd}and 3^{rd} in the 10 iterations (runs) when the foundation model uses maximum input length of 512 bytes. Median values of the rankings from the 10 iterations are also shown, e.g., median ranking for “ZG16” is 1.5, median ranking for “FCGBP” is 2, and median ranking for “MUC2” is 2, when the foundation model uses maximum input length of 512 bytes.

Similarly, the use case example shows results obtained from ten iterations of the foundation model using 256 bytes as maximum input length and results obtained from ten iterations of the foundation model using 128 bytes as maximum input length. The standard deviation of median rankings for runs with 512, 256 and 128 byte sizes as maximum input length are computed and shown. For instance, for “ZG16” gene, the standard deviation of the median rankings is 0.3; for “FCGBP” gene, the standard deviation of the median rankings is 0.3; and for “MUC2” gene, the standard deviation of the median rankings is 0.6. The standard deviation can be compared with a predefined threshold standard deviation, e.g., 1.0. If the computed standard deviation is less that the predefined threshold standard deviation, it can be considered that there is consistent interpretability among the rankings produced using different maximum input length. In this use case example, if only the top 3 ranked genes are considered, since the standard deviations are less than the predefined threshold, the input maximum length of 128 can be used as the configuration parameter, i.e., the shortest maximum length that can provide for savings in processing time and memory (storage) space. In this use case example, the foundation model ran on a 16 core processor. Using a different processor configuration may also alter the results. Based on such results, the system and/or method may recommend an appropriate processor configuration as well.

Briefly, the median is the middle value of an ordered data set. The first quartile (Q1) is the median of the lower half of the data set. The third quartile (Q3) is the median of the upper half of the data set. FIG. 3 shows the median, Q1 and Q3 for the results of iterations ran with different maximum input length configuration parameters.

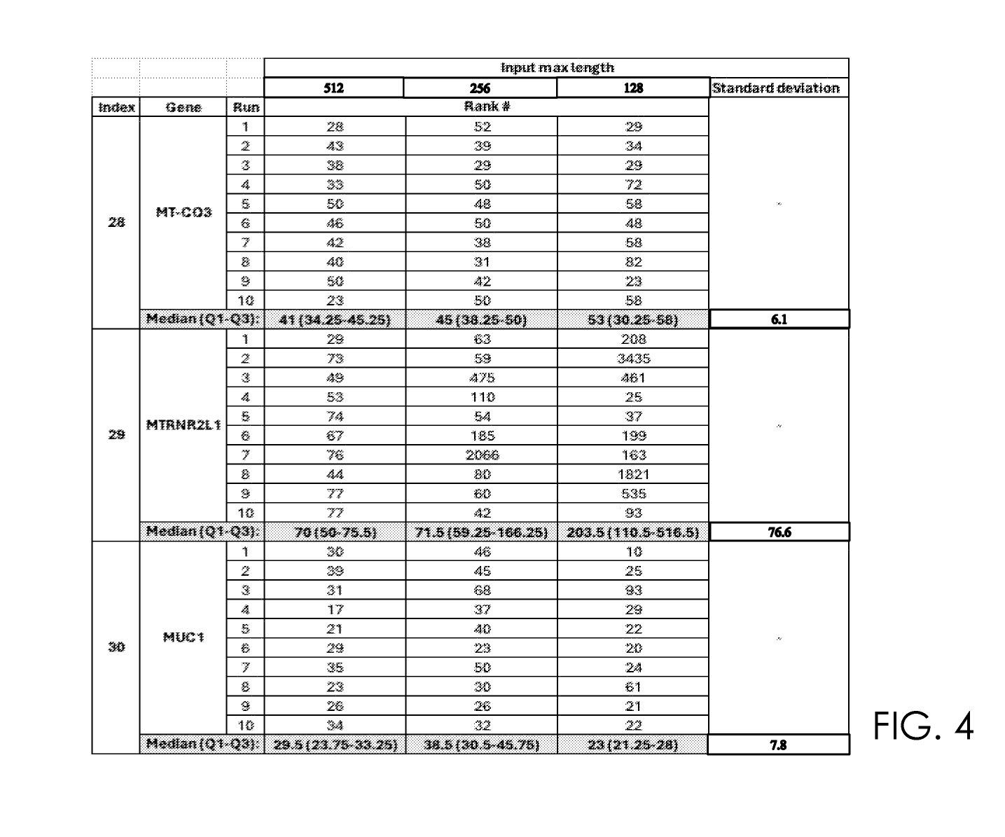

FIG. 4 shows a continuation of the use case shown in FIG. 3, where the top 28^{th}, 29^{th} and 30^{th} ranked genes are shown. For “MT-CO3” gene, the rankings over the 10 iterations vary from 28 to 50, resulting in a median ranking of 41 when 512 byte size is used as configuration parameter for the maximum input length. For “MTRNR2L1” gene, the rankings over the 10 iterations vary from 29 to 77, resulting in a median ranking of 41 when 512 byte size is used as configuration parameter for the maximum input length. For “MUC1” gene, the rankings over the 10 iterations vary from 30 to 39, resulting in a median ranking of 29.5when 512 byte size is used as configuration parameter for the maximum input length. Similarly, the use case example shows results obtained from ten iterations of the foundation model using 256 bytes as maximum input length and results obtained from ten iterations of the foundation model using 128 bytes as maximum input length. The standard deviation of median rankings for runs with 512, 256 and 128 byte sizes as maximum input length are computed and shown. For instance, for “MT-CO3” gene, the standard deviation of the median rankings is 0.1; for “MTRNR2L1” gene, the standard deviation of the median rankings is 76.6; and for “MUC1” gene, the standard deviation of the median rankings is 7.8. By way of example, consider that a predefined standard deviation threshold is 15. Because the computed standard deviation is greater than the predefined threshold standard deviation, the system and/or method determines that using 128 bytes size as the maximum input length creates inconsistent interpretability. In this example, thus, the system and/or method may recommend 256 bytes size as the maximum input length, e.g., since the deviation between the results of using 512 and 256 bytes sizes as the maximum input length is within the predefined standard deviation threshold.

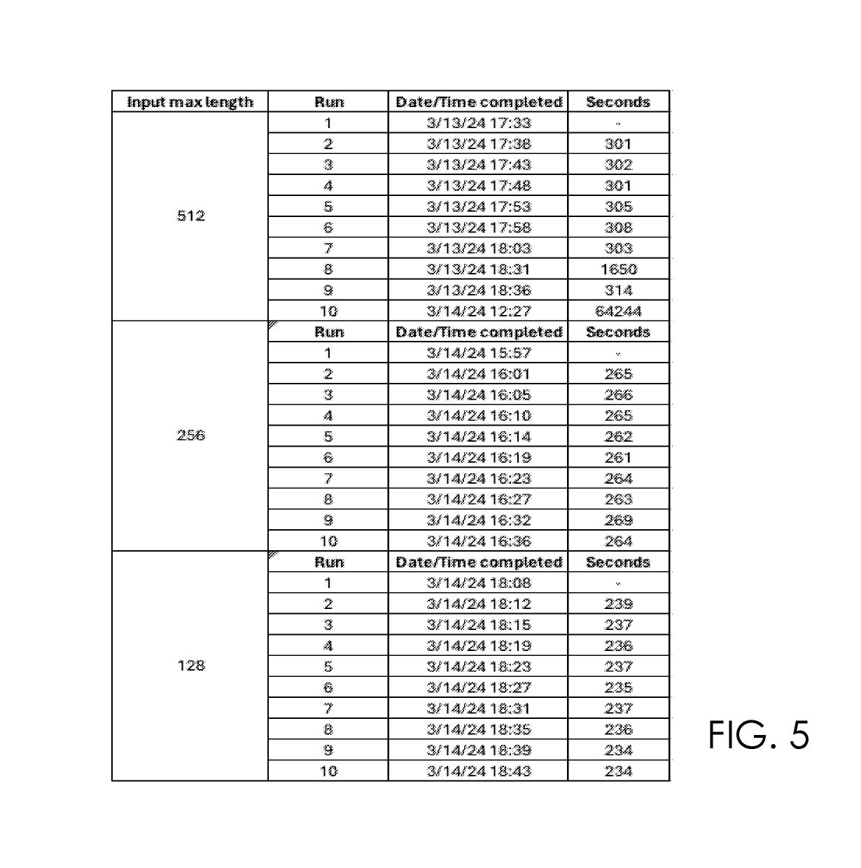

FIG. 5 shows processing time results from use case examples where different byte sizes are used as maximum input length in runs a foundation model. Median processing time in seconds is 13% less for maximum input length 256 bytes than 512 bytes; Median processing time in seconds is 22% less for maximum input length 128 bytes than 512 bytes. This example illustrates that using a shorter maximum input length saves processing time, while also having consistent interpretability in the results.

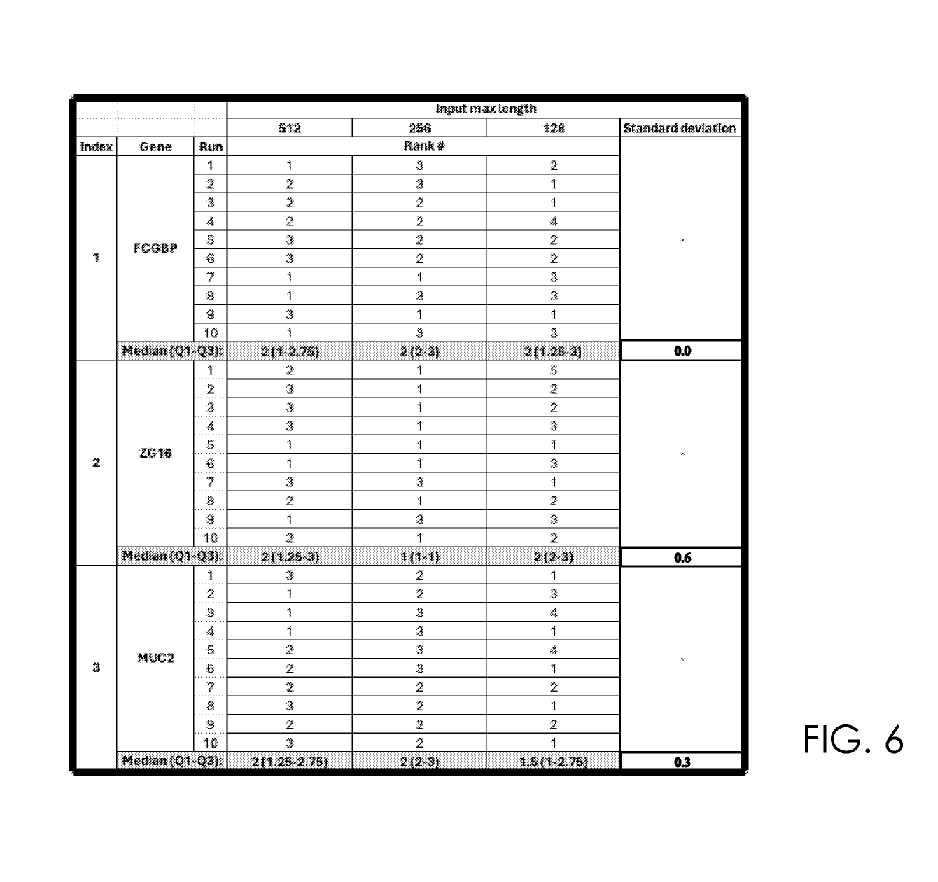

FIG. 6 shows another use case example, in which an 8 core processor is used in running the foundation model in some embodiments. Results are obtained using the similar process performed in obtaining the results shown in FIGS. 3, 4 and 5. In this use case example, the standard deviation for the 1^{st} ranked gene is 0.0; the standard deviation for the 2^{nd} ranked gene is 0.6; and the standard deviation for the 3^{rd} ranked gene is 0.0. Since the standard deviations are less than the predefined threshold, e.g., 1.0, the system and/or method considers that there is consistent interpretability throughout the runs regardless of the size of the maximum input length.

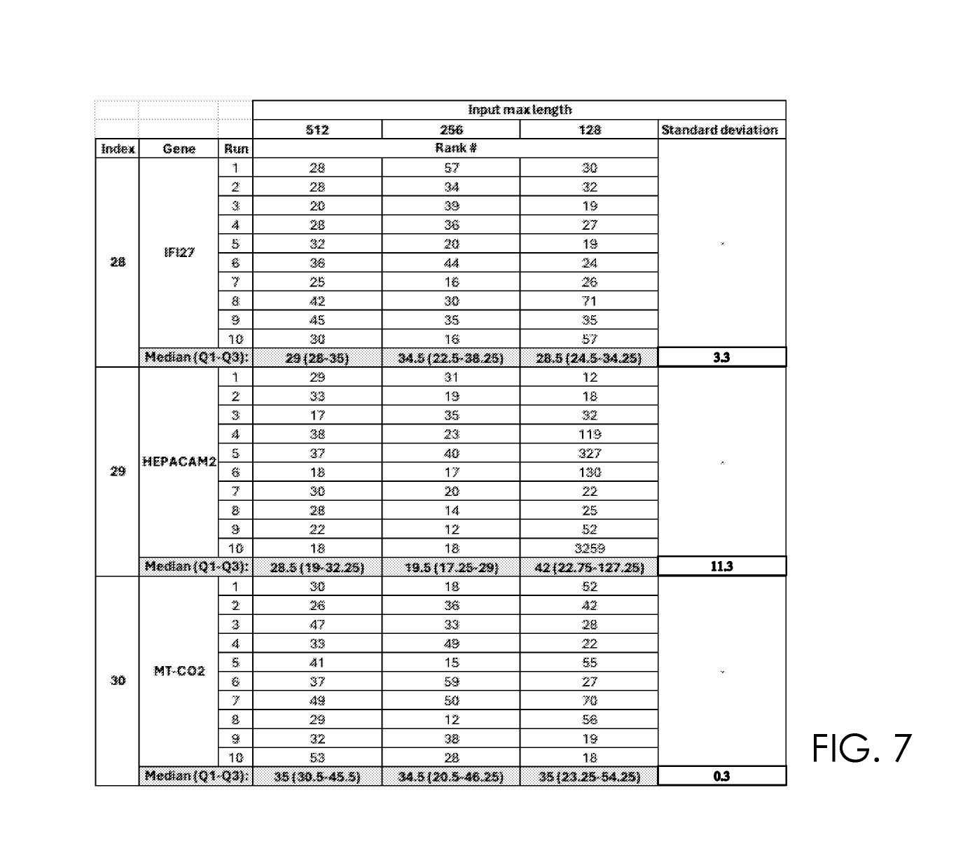

FIG. 7 shows continuation of the use case example of FIG. 6, where the results are shown for the 28^{th}, 29^{th} and 30^{th} ranked genes. Based on the standard deviation computed for each of the 28^{th}, 29^{th} and 30^{th} ranked genes among the ten iterations, the system and/or method determines the size of the maximum input length to configure and present to the foundation model.

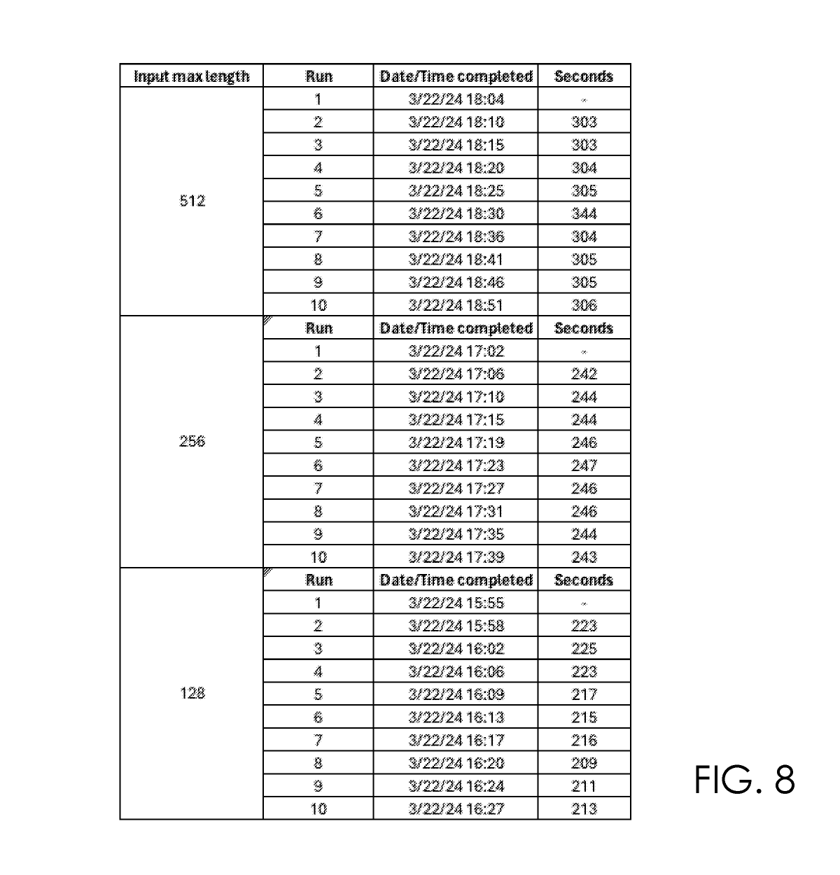

FIG. 8 shows processing time results from use case examples where different byte sizes are used as maximum input length in runs a foundation model in example use case shown in FIGS. 6 and 7. Median processing time in seconds is 20% less for maximum input length of 256 bytes than 512 bytes; Median processing time in seconds is 29% less for maximum input length 128 bytes than 512 bytes. This example also illustrates that using a shorter maximum input length saves processing time, while also having consistent interpretability in the results.

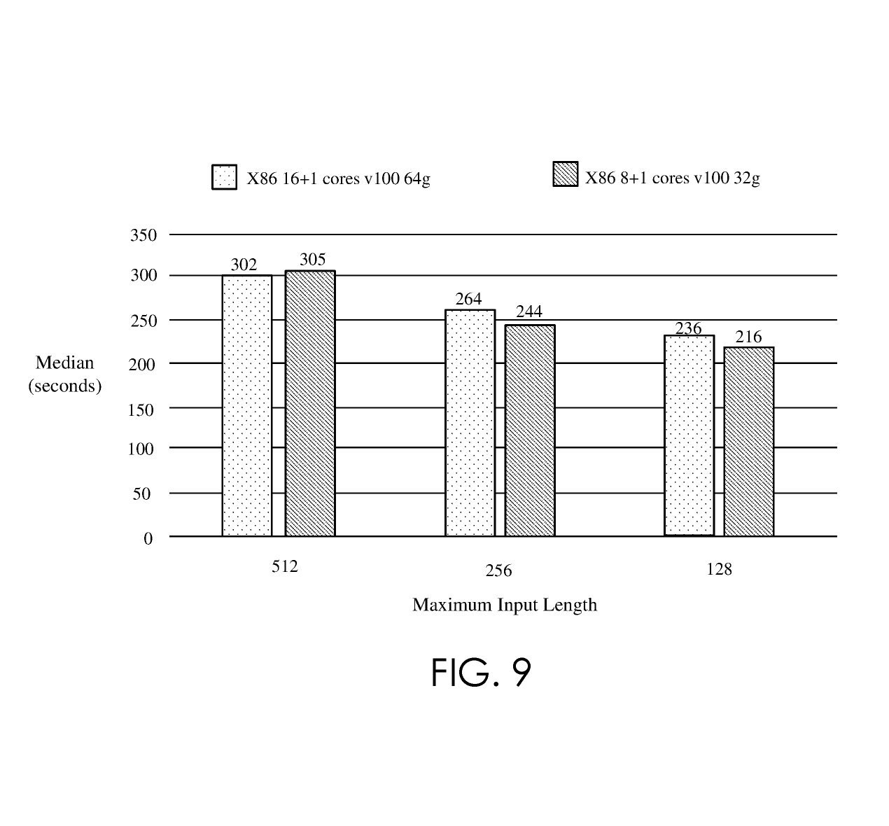

FIG. 9 shows median processing time comparison between running the foundation model on different types of processors (e.g., x86 16+1 cores v100 64g and x86 8+1 cores v100 32g) with different maximum input length configurations, e.g., 512, 256 and 128 bytes. For instance, median processing time in seconds for 512 maximum input length for the foundation model running on x86 16+1 cores v100 64g is 302; median processing time in seconds for 512 maximum input length for the foundation model running on X86 8+1 cores v100 32g is 305; median processing time in seconds for 256 maximum input length for the foundation model running on x86 16+1 cores v100 64g is 264; median processing time in seconds for 256 maximum input length for the foundation model running on X86 8+1 cores v100 32g is 244; median processing time in seconds for 128 maximum input length for the foundation model running on x86 16+1 cores v100 64g is 236; median processing time in seconds for 128 maximum input length for the foundation model running on X86 8+1 cores v100 32g is 216. Given the results, the system and/or method in some embodiments chooses the configuration that results in the least processing time that still provides consistent interpretability.

As described herein, the system and/or method uses an iterative process that can determine configuration parameters in a single cell genetic analysis. The system and/or method in some embodiments can provide for shorter processing times (measured by time, e.g., seconds), while providing a similar level of interpretability (measured by discrepancy from a standard deviation). For example, low standard deviations of gene rankings using different configurations prove that the model results did not suffer (e.g., have consistent interpretability). For instance, non-optimal parameter configuration may result in the interpretability process breaking. Further, more extensive hardware does not necessarily yield better performance.

In some embodiments, a method to optimize values of configuration parameters of a foundation model in a single cell genetic analysis, includes iteratively evaluating different sets of values of configuration parameters. For example, a processor set performing the method applies different values as a configuration parameter. Based on the different values of the configuration parameter, the processor set performs an interpretability assessment. For instance, by running a foundation model or a biomedical foundation model using a value of the configuration parameter, the processor set calculates each gene association strength score value on a cell. The processor set sorts or ranks the genes in a descending order according to their association strength score values. The processor set selects a first set of genes that includes a pre-defined number of top ranked genes, and also selects a second set of genes that includes a pre-defined number of additional genes. The processor set stores the two sets of genes, i.e., the first set and the second set of genes. The processor set repeats the process a pre-defined number of times considering the two sets of selected genes, and in each iteration stores the rankings of the genes and the association strength scores. For each gene of the two sets, the processor set calculates a median rank considering the total number of iterations performed. The above process is repeated for the different values of the configuration parameter. For instance, the above process is repeated for each of the different values used as the configuration parameter. For each configuration parameter, and for each gene, the processor set calculates a standard deviation value of the medians, and compares the standard deviation value to a pre-defined threshold value. The processor set also calculates the median time to complete processing the iterations. Based on discrepancy from the standard deviation threshold and processing time improvement (processing times of different iterations using different values of the configuration parameter), the processor set determines final value of the configuration parameter value. In some embodiments, Note that in one embodiment more than two sets of genes may be evaluated. Note also that in one embodiment a one set of genes may be evaluated. A set of genes may be any number of genes (e.g., 1 or more). Note that in one embodiment the order of the genes used for evaluation may be of any order, not necessary, 1^{st}, 2^{nd} ,3^{rd}, etc. The genes may be selected at random, or in any order out of the entire range of gene types. Note also that in one embodiment specific genes may be pre-selected for evaluation, either by a human or by a computational method.

In some embodiments, a set of configuration parameters are evaluated by using a set of values for those configuration parameters at each iteration, resulting in discovering a combination of configuration parameters (rather than only one). In some embodiments, the configuration parameters include one or more of pad to multiple (e.g., 8, 16, 32), maximum input length (e.g., 128, 256, 512), sequence order (e.g., none, random) and batch size (e.g., 64). In some embodiments, the pre-defined number of top ranked genes can be any integer value such as 3 or any other integer. In some embodiments, the pre-defined number of additional genes can be any integer value such as 3 or any other integer. In some embodiments, the pre-defined standard deviation threshold can be greater than or equal to zero, or any positive value. In some embodiments, additional parameters such as current or anticipated bandwidth or other usage criteria of computational resources affecting performance of the available computational resources (e.g., a random day, a holiday, a day close to a submission deadline to a conference, and/or others) can be considered.

In some embodiments, the system and/or method assesses whether an improved performance results in a decreased interpretability in a single cell genetic analysis. In some embodiments, improved performance is measured by one or more of, reduced processing time, reduced incidences of code breaks and/or disconnection from network. In some embodiments, interpretability difference is measured by recurrent conclusion (e.g., genes are associated to the same extent to a cell even if computational resources are reduced). For example, interpretability difference among results produced by using different sets of configuration parameters can be measured based on a standard deviation of the results. In some embodiments, the system and/or method is applicative to single cell genetic analysis, and the system and/or method considers all genes (e.g., without a notion of reduction of the number of genes to analyze).

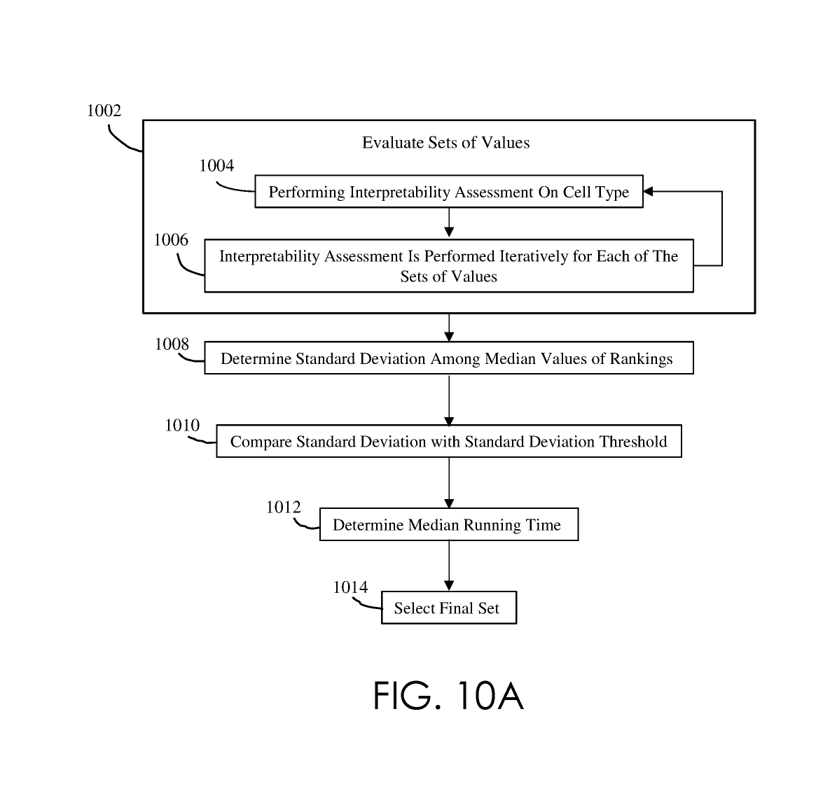

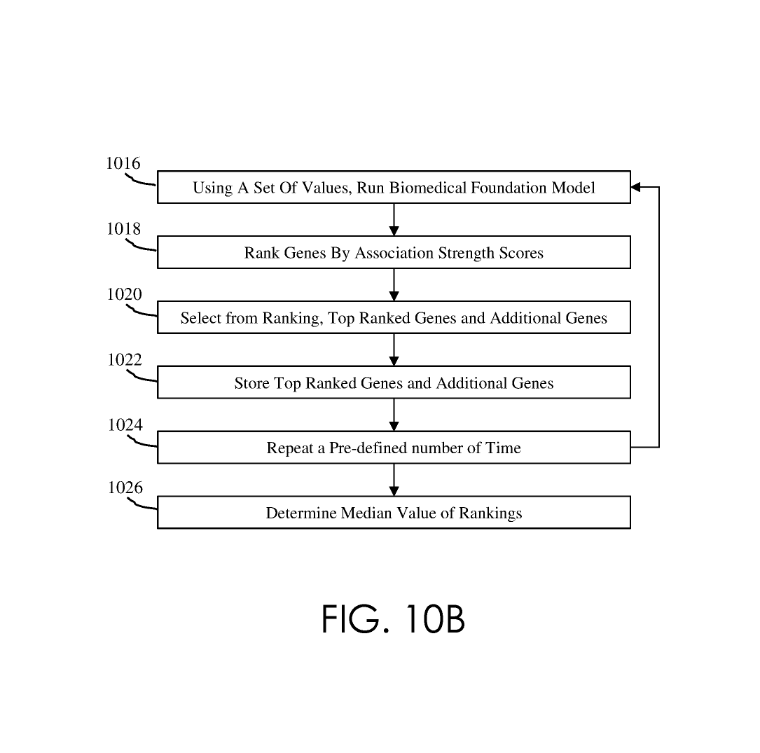

FIGS. 10A-10B show flow diagrams illustrating a method of optimizing configuration of input parameters to a biomedical foundation model in some embodiments. The method can be performed on or by one or more computer processors or hardware processors. At 1002, the method evaluates sets of values for a set of configuration parameters of a biomedical foundation model. In some embodiments, the evaluation includes performing an interpretability assessment on a cell type. In some embodiments, the set of configuration parameters includes at least one configuration parameter. In some embodiments, the set of configuration parameters includes pad to multiple. In some embodiments, the set of configuration parameters includes maximum input length. In some embodiments, the set of configuration parameters includes sequence order. In some embodiments, the set of configuration parameters includes batch size.

Performing the interpretability assessment is shown in FIG. 10B. Referring to FIG. 10B, at 1016, using a set of values from the sets of values as the set of configuration parameters for the biomedical foundation model, the method runs the biomedical foundation model to obtain association strength scores between the cell type and genes. At 1018, the method ranks the genes by the association strength scores. In some embodiments, ranking the genes by the association strength scores includes sorting the genes in descending order according to an association strength score of each of the genes.

At 1020, the method selects from the ranking, a pre-defined number of top ranked genes and a pre-defined number of additional genes. At 1022, the method stores the pre-defined number of top ranked genes and the pre-defined number of additional genes. The method repeats, as shown at 1024, by a pre-defined number of times, the using the set of values and the ranking in consideration with the pre-defined number of top ranked genes and the pre-defined number of additional genes. At 1026, for each gene in the pre-defined number of top ranked genes and the pre-defined number of additional genes, the method determines a median value of rankings determined from the repeating of the using and the ranking the pre-defined number of times.

Referring back to FIG. 10A, the method performs the interpretability assessment iteratively for each of the sets of values for the set of configuration parameters, as shown at 1006. At 1008, for each gene in the pre-defined number of top ranked genes and the pre-defined number of additional genes, the method determines a standard deviation among median values of rankings that are computed during the interpretability assessment performed iteratively. At 1010, the method compares the standard deviation with a standard deviation threshold. At 1012, the method determines a median running time of the biomedical foundation model for each iteration of the interpretability assessment. At 1014, the method, based on the comparison of the standard deviation with the standard deviation threshold and the median running time, selects a final set from the sets of values to use as the set of configuration parameters for running the biomedical foundation model. In some embodiments, the final set is selected based on the standard deviation meeting the standard deviation threshold and the median running time meeting an improvement criterion.

The terminology used herein is for the purpose of describing particular embodiments only and is not intended to be limiting of the invention. As used herein, the singular forms "a", "an" and "the" are intended to include the plural forms as well, unless the context clearly indicates otherwise. As used herein, the term “or” is an inclusive operator and can mean “and/or”, unless the context explicitly or clearly indicates otherwise. It will be further understood that the terms "comprise", "comprises", "comprising", "include", "includes", "including", and/or "having," when used herein, can specify the presence of stated features, integers, steps, operations, elements, and/or components, but do not preclude the presence or addition of one or more other features, integers, steps, operations, elements, components, and/or groups thereof. As used herein, the phrase “in some embodiments” does not necessarily refer to the same embodiment, although it may. As used herein, the phrase “in one embodiment” does not necessarily refer to the same embodiment, although it may. As used herein, the phrase “in another embodiment” does not necessarily refer to a different embodiment, although it may. Further, embodiments and/or components of embodiments can be freely combined with each other unless they are mutually exclusive.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A computer-implemented method comprising:
evaluating sets of values for a set of configuration parameters of a biomedical foundation model, the evaluating comprising, performing an interpretability assessment on a cell type, the interpretability assessment comprising:
using a set of values from the sets of values as the set of configuration parameters for the biomedical foundation model, running the biomedical foundation model to obtain association strength scores between the cell type and genes;
ranking the genes by the association strength scores;
selecting from the ranking, a pre-defined number of top ranked genes and a pre-defined number of additional genes;
storing the pre-defined number of top ranked genes and the pre-defined number of additional genes;
repeating by a pre-defined number of times, the using and the ranking in consideration with the pre-defined number of top ranked genes and the pre-defined number of additional genes; and
for each gene in the pre-defined number of top ranked genes and the pre-defined number of additional genes, determining a median value of rankings determined from the repeating of the using and the ranking the pre-defined number of times;
wherein the interpretability assessment is performed iteratively for each of the sets of values for the set of configuration parameters;
for each gene in the pre-defined number of top ranked genes and the pre-defined number of additional genes, determining a standard deviation among median values of rankings that are computed during the interpretability assessment performed iteratively;
comparing the standard deviation with a standard deviation threshold;
determining a median running time of the biomedical foundation model for each iteration of the interpretability assessment; and
based on the comparison of the standard deviation with the standard deviation threshold and the median running time, selecting a final set from the sets of values to use as the set of configuration parameters for running the biomedical foundation model.

2. The computer-implemented method of claim 1, wherein the ranking the genes by the association strength scores includes sorting the genes in descending order according to an association strength score of each of the genes.

3. The computer-implemented method of claim 1, wherein the final set is selected based on the standard deviation meeting the standard deviation threshold and the median running time meeting an improvement criterion.

4. The computer-implemented method of claim 1, wherein the set of configuration parameters includes at least one configuration parameter.

5. The computer-implemented method of claim 4, wherein the set of configuration parameters includes pad to multiple.

6. The computer-implemented method of claim 4, wherein the set of configuration parameters includes maximum input length.

7. The computer-implemented method of claim 4, wherein the set of configuration parameters includes sequence order.

8. The computer-implemented method of claim 4, wherein the set of configuration parameters includes batch size.

9. The computer-implemented method of claim 1, wherein the pre-defined number of top ranked genes is three.

10. The computer-implemented method of claim 1, wherein the pre-defined number of additional genes is three.

11. A computer program product comprising:
one or more computer-readable storage media; and
program instructions stored on the one or more storage media to perform operations comprising:
evaluating sets of values for a set of configuration parameters of a biomedical foundation model, the evaluating comprising, performing an interpretability assessment on a cell type, the interpretability assessment comprising:
using a set of values from the sets of values as the set of configuration parameters for the biomedical foundation model, running the biomedical foundation model to obtain association strength scores between the cell type and genes;
ranking the genes by the association strength scores;
selecting from the ranking, a pre-defined number of top ranked genes and a pre-defined number of additional genes;
storing the pre-defined number of top ranked genes and the pre-defined number of additional genes;
repeating by a pre-defined number of times, the using and the ranking in consideration with the pre-defined number of top ranked genes and the pre-defined number of additional genes; and
for each gene in the pre-defined number of top ranked genes and the pre-defined number of additional genes, determining a median value of rankings determined from the repeating of the using and the ranking the pre-defined number of times;
wherein the interpretability assessment is performed iteratively for each of the sets of values for the set of configuration parameters;
for each gene in the pre-defined number of top ranked genes and the pre-defined number of additional genes, determining a standard deviation among median values of rankings that are computed during the interpretability assessment performed iteratively;
comparing the standard deviation with a standard deviation threshold;
determining a median running time of the biomedical foundation model for each iteration of the interpretability assessment; and
based on the comparison of the standard deviation with the standard deviation threshold and the median running time, selecting a final set from the sets of values to use as the set of configuration parameters for running the biomedical foundation model.

12. The computer program product of claim 11, wherein the ranking the genes by the association strength scores includes sorting the genes in descending order according to an association strength score of each of the genes.

13. The computer program product of claim 11, wherein the final set is selected based on the standard deviation meeting the standard deviation threshold and the median running time meeting an improvement criterion.

14. The computer program product of claim 11, wherein the set of configuration parameters includes at least one configuration parameter.

15. The computer program product of claim 14, wherein the set of configuration parameters includes pad to multiple.

16. The computer program product of claim 14, wherein the set of configuration parameters includes maximum input length.

17. The computer program product of claim 14, wherein the set of configuration parameters includes sequence order.

18. The computer program product of claim 14, wherein the set of configuration parameters includes batch size.

19. The computer program product of claim 11, wherein the pre-defined number of top ranked genes is three.

20. A computer system comprising:
a processor set;
a set of one or more computer-readable storage media;
program instructions, collectively stored in the set of one or more computer-readable storage media, for causing the processor set to perform the following computer operations:
evaluating sets of values for a set of configuration parameters of a biomedical foundation model, the evaluating comprising, performing an interpretability assessment on a cell type, the interpretability assessment comprising:
using a set of values from the sets of values as the set of configuration parameters for the biomedical foundation model, running the biomedical foundation model to obtain association strength scores between the cell type and genes;
ranking the genes by the association strength scores;
selecting from the ranking, a pre-defined number of top ranked genes and a pre-defined number of additional genes;
storing the pre-defined number of top ranked genes and the pre-defined number of additional genes;
repeating by a pre-defined number of times, the using and the ranking in consideration with the pre-defined number of top ranked genes and the pre-defined number of additional genes; and
for each gene in the pre-defined number of top ranked genes and the pre-defined number of additional genes, determining a median value of rankings determined from the repeating of the using and the ranking the pre-defined number of times;
wherein the interpretability assessment is performed iteratively for each of the sets of values for the set of configuration parameters;
for each gene in the pre-defined number of top ranked genes and the pre-defined number of additional genes, determining a standard deviation among median values of rankings that are computed during the interpretability assessment performed iteratively;
comparing the standard deviation with a standard deviation threshold;
determining a median running time of the biomedical foundation model for each iteration of the interpretability assessment; and
based on the comparison of the standard deviation with the standard deviation threshold and the median running time, selecting a final set from the sets of values to use as the set of configuration parameters for running the biomedical foundation model.
